(19) United States
(12) Patent Application Publication
(10) Pub. No.: US 2013/0082722 A1
(43) Pub. Date: Apr. 4, 2013

(54) METHOD OF FORMING NANOPORE AND STRUCTURE FORMED WITH NANOPORE

(71) Applicant: LG ELECTRONICS INC., Seoul (KR)

(72) Inventors: Da Mi KIM, Seoul (KR); Jean Kun OH, Seoul (KR); Paul HAN, Seoul (KR); Yong SHIN, Seoul (KR); Bong Chu SHIM, Seoul (KR); Ji Tae KIM, Seoul (KR)

(73) Assignee: LG ELECTRONICS INC., Seoul (KR)

(21) Appl. No.: 13/626,004

(22) Filed: Sep. 25, 2012

(30) Foreign Application Priority Data
Sep. 30, 2011 (KR) 10-2011-0099787

(51) Int. Cl. C07H 21/00; G01N 27/04; C12P 19/34

(52) U.S. Cl. 324/692; 536/25.3; 435/91.5

(57) ABSTRACT

The present invention relates to a method of forming a nanopore and a structure formed with the nanopore. The present invention relates to a method of forming a nanopore by preparing a first structure and a second structure having a surface on which nucleotides can be attached; attaching one ends of a plurality of oligonucleotides complementary to each other on the surface; binding the first structure and the second structure; and removing some of the bound oligonucleotides. The present invention is effective in that a pore of a desired size can be accurately formed by adjusting the length of the oligonucleotides.

### BACKGROUND OF THE INVENTION

1. Field of the Invention

The present invention relates to a method of forming a pore for detecting or analyzing a target material existing in a sample and a structure formed with the pore, and particularly, to a method of forming a pore by adjusting the size of the pore as desired.

2. Background of the Related Art

A variety of methods have been developed to detect target bio-molecules in a sample, and a method using a nanopore among the methods is a bio-pore mimetic system, which is spotlighted as a high-sensitive DNA detection system.

There are various DNA detection systems using a nanopore. For example, the object of U.S. Pat. No. 6,015,714 (Title of the invention: Characterization of individual polymer molecules based on monomer-interface interactions) is to perform DNA sequencing by distinguishing each base configuring DNA using a very sensitive signal that the nanopore has. There are two pools, and a small pore which allows DNA to enter one by one is provided between the pools. A DNA biopolymer is loaded on either of the pools, and the DNA sequencing is accomplished by measuring the DNA biopolymer passing through the pore.

U.S. Pat. No. 6,362,002 (Title of the invention: Characterization of individual polymer molecules based on monomer interface interactions) discloses a method of forming a nanopore through which bases of single stranded DNA sequentially pass and distinguishing a double stranded nucleic acid from a single stranded nucleic acid (the double stranded nucleic acid passes after being dissociated into single stranded nucleic acids, and thus it takes time).

U.S. Laid-opened Patent No. 2003/0104428 (Title of the invention: Method for characterization of nucleic acid molecules) discloses a technique for detecting a specific base sequence of DNA by grasping a specific sequence using a different material, protein or DNA for recognizing a specified local area of DNA, and observing changes in signal caused by the different material bound to the DNA, in order to grasp characteristics of sample DNA using a nanopore.

U.S. Pat. No. 6,428,959 (Title of the invention: Methods of Determining the presence of double stranded nucleic acids in a sample) discloses a method of distinguishing a double stranded nucleic acid from a single stranded nucleic acid through a blockade of current by measuring amplitude of the current flowing through a nanopore while passing a nucleic acid in a fluid sample through the nanopore having a diameter of 3 to 6 nm.

However, in such conventional methods and apparatuses for detecting DNA using a nanopore, the diameter of the nanopore is large, and thus resolution thereof is low. In addition, since the diameter of a required nanopore should be less than 10 nm, preferably less than 5 nm, the structure of the apparatus for detecting DNA and detecting conditions thereof are very complicated.

Although a lot of efforts have been made until present in order to fabricate a nanopore having a diameter as small as a bio-pore, there are a lot of problems since the nanopore is difficult to fabricate in reality.

### SUMMARY OF THE INVENTION

Therefore, the present invention has been made in view of the above problems, and it is an object of the present invention to form a pore of a desired size in a simple and easy way.

To accomplish the above object, according to one aspect of the present invention, there is provided a method of forming a nanopore, the method including the steps of: preparing a first structure and a second structure having a surface on which nucleotides can be attached; attaching one ends of a plurality of oligonucleotides on the surface of the prepared first structure, and attaching one ends of a plurality of oligonucleotides having all or some areas complementary to each of the plurality of oligonucleotides on the surface of the prepared second structure; and binding the plurality of oligonucleotides attached to the first structure and the second structure to each other.

That is, the present invention may form a periphery surrounding a pore by binding oligonucleotides having some areas complementary to each other among the plurality of oligonucleotides attached to the first structure and the second structure and form a pore center by oligonucleotides uncomplementary to each other or oligonucleotides that are not bound to each other although they are complementary to each other.

As a specific example, the step of attaching includes the steps of attaching one ends of a plurality of oligonucleotides on the surface of the prepared first structure, and attaching one ends of a plurality of oligonucleotides having some areas complementary to each of the plurality of oligonucleotides on the surface of the prepared second structure, and after the step of binding each other, the step of removing some oligonucleotides among the plurality of oligonucleotides bound to each other may be further provided. That is, the method of forming a nanopore of the present invention may include the steps of: preparing a first structure and a second structure having a surface on which nucleotides can be attached; attaching one ends of a plurality of oligonucleotides on the surface of the prepared first structure, and attaching a plurality of oligonucleotides having some areas complementary to each of the plurality of oligonucleotides on the surface of the prepared second structure; binding the plurality of oligonucleotides attached to the first structure and the second structure to each other, and removing some oligonucleotides among the plurality of oligonucleotides bound to each other.

Here, some oligonucleotides among the plurality of oligonucleotides include bases of artificial sequence that can be cut by a specific enzyme in the oligonucleotides.

In addition, as a specific example, the step of attaching includes the steps of attaching one ends of a plurality of oligonucleotides on the surface of the prepared first structure, and attaching a plurality of oligonucleotides having all or some areas complementary to each of some oligonucleotides among the plurality of oligonucleotides and one or more oligonucleotides uncomplementary to oligonucleotides positioned between the some oligonucleotides on the surface of the prepared second structure, and the step of binding to each other is the step of binding the complementary oligonucleotides among the plurality of oligonucleotides attached to the first structure and the second structure to each other. That is, the method of forming a nanopore of the present invention may include the steps of: preparing a first structure and a second structure having a surface on which nucleotides can be attached; attaching one ends of a plurality of oligonucleotides on the surface of the prepared first structure, and attaching a plurality of oligonucleotides having all or some areas complementary to each of some oligonucleotides among the plurality of oligonucleotides and one or more oligonucleotides uncomplementary to oligonucleotides positioned between the some oligonucleotides on the surface of the prepared second structure; and binding the complementary oligonucleotides among the plurality of oligonucleotides attached to the first structure and the second structure to each other.

Here, the step of binding oligonucleotides to each other is binding oligonucleotides complementary to each other among the plurality of oligonucleotides attached to the first structure and the second structure, and forming a pore by the one or more uncomplementary oligonucleotides.

As a specific example, the step of attaching includes the steps of attaching one ends of a plurality of oligonucleotides at positions spaced apart from each other on one surface of the prepared structure, and attaching a plurality of oligonucleotides having all or some areas complementary to each of the plurality of oligonucleotides on one surface of the prepared second structure corresponding to the positions spaced apart from each other on the first structure. That is, the method of forming a nanopore of the present invention may include the steps of: preparing a first structure and a second structure having a surface on which nucleotides can be attached; attaching one ends of a plurality of oligonucleotides at positions spaced apart from each other on one surface of the prepared first structure, and attaching a plurality of oligonucleotides having all or some areas complementary to each of the plurality of oligonucleotides on one surface of the prepared second structure corresponding to the positions spaced apart from each other on the first structure; and binding the plurality of oligonucleotides attached to the first structure and the second structure to each other.

Here, the step of binding the oligonucleotides to each other is binding oligonucleotides complementary to each other among the plurality of oligonucleotides attached to the first structure and the second structure and forming the pore at the spaced position.

On the other hand, in another embodiment of the present invention, there is provided a structure formed with a nanopore, the structure comprising: a first structure having a surface attached with one ends of a plurality of oligonucleotides; a second structure having a surface attached with a plurality of oligonucleotides having all or some areas complementary to each of the plurality of oligonucleotides; and a pore formed by binding the plurality of oligonucleotides attached to the first structure and the second structure.

That is, the present invention may form a periphery surrounding a pore by binding oligonucleotides having some areas complementary to each other among the plurality of oligonucleotides attached to the first structure and the second structure and form a pore center by oligonucleotides uncomplementary to each other or oligonucleotides that are not bound to each other although they are complementary to each other.

As a specific example, in the structure according to the present invention, the plurality of oligonucleotides attached to the first structure and the second structure is bound to each other, and a pore is formed by removing some of the bound oligonucleotides. That is, the present invention may be a structure formed with a nanopore, the structure including a first structure having a surface attached with one ends of a plurality of oligonucleotides; a second structure having a surface attached with a plurality of oligonucleotides having all or some areas complementary to each of the plurality of oligonucleotides; and a pore formed by binding the plurality of oligonucleotides attached to the first structure and the second structure to each other and removing some of the bound oligonucleotides.

Here, the plurality of oligonucleotides attached to the first structure and the plurality of oligonucleotides attached to the second structure are preferably attached at positions corresponding to each other.

As a specific example, in the present invention, the second structure has a surface attached with a plurality of oligonucleotides having all or some areas complementary to each of some oligonucleotides among the plurality of oligonucleotides and one or more oligonucleotides uncomplementary to oligonucleotides positioned between the some oligonucleotides, and the pore is formed by the one or more uncomplementary oligonucleotides, while the complementary oligonucleotides among the plurality of oligonucleotides attached to the first structure and the second structure are bound to each other. That is, the structure formed with a nanopore of the present invention may include a first structure having a surface attached with one ends of a plurality of oligonucleotides; a second structure having a surface attached with a plurality of oligonucleotides having all or some areas complementary to each of some oligonucleotides among the plurality of oligonucleotides and one or more oligonucleotides uncomplementary to oligonucleotides positioned between the some oligonucleotides; and a pore formed by one or more uncomplementary oligonucleotides, in which the complementary oligonucleotides among the plurality of oligonucleotides attached to the first structure and the second structure are bound to each other.

Here, the one or more uncomplementary oligonucleotides are preferably positioned between the complementary oligonucleotides attached to the second structure.

As a specific example, in the present invention, the first structure has one surface attached with one ends of a plurality of oligonucleotides at positions spaced apart from each other, and the second structure may have a surface attached with a plurality of oligonucleotides having all or some areas complementary to each of the plurality of oligonucleotides at positions corresponding to the positions spaced apart from each other on the first structure. That is, the structure formed with a nanopore of the present invention may include a first structure having a surface attached with one ends of a plurality of oligonucleotides at positions spaced apart from each other; a second structure having a surface attached with a plurality of oligonucleotides having all or some areas complementary to each of the plurality of oligonucleotides at positions corresponding to the positions spaced apart from each other on the first structure; and a pore formed by binding the plurality of oligonucleotides attached to the first structure and the second structure.

Here, the plurality of oligonucleotides attached to the first structure and the second structure is bound to each other, and the pore is preferably formed between the positions spaced apart from each other.

On the other hand, in still another embodiment of the present invention, there is provided a nucleic acid molecule detection apparatus using a nanopore, the apparatus comprising: a structure formed with the nanopore; an electrode for applying voltage to the nanopore of the structure; and a measurement unit for measuring an electrical signal generated when a sample containing DNA passes through the nanopore.

Specifics of the other embodiments are included in the detailed descriptions and figures.

### BRIEF DESCRIPTION OF THE DRAWINGS

FIG. 1 is a cross-sectional view showing a structure having a surface on which nucleotides can be attached according to the present invention.

FIG. 2 is a plan view showing a plurality of oligonucleotides attached on the surface of a structure according to the present invention.

FIG. 3 shows an enlarged view of the plurality of oligonucleotides in FIG. 2.

FIG. 4 is a mimetic view showing a plurality of oligonucleotides attached on the surface of a structure in a state of being bound to each other according to the present invention.

FIG. 5 is a mimetic view showing a process of forming a pore by removing some of a plurality of oligonucleotides bound to each other according to the present invention.

FIG. 6 is a mimetic view showing some oligonucleotides uncomplementary to each other in a state of being attached on the surface of a structure according to the present invention.

FIG. 7 is a mimetic view showing a process of forming a pore by the oligonucleotides of FIG. 6.

FIG. 8 is a mimetic view showing oligonucleotides complementary to each other in a state of being attached at positions apart from each other on the surface of a structure according to the present invention.

FIG. 9 is a mimetic view showing a process of forming a pore by the oligonucleotides of FIG. 8.

FIG. 10 is a cross-sectional view showing a process of sequencing target DNA using a structure formed with a pore according to an embodiment of the present invention.

FIG. 11 is a perspective view of FIG. 10.

FIG. 12 is a cross-sectional view showing a silicon structure having a gold surface on which nucleotides can be attached according to the present invention.

FIG. 13 is a cross-sectional view showing DNA oligonucleotides attached to the structure of FIG. 12.

FIG. 14 is a perspective view showing a process of sequencing target DNA using the structure of FIG. 12.

FIG. 15 is a cross-sectional view showing a structure formed with a gold surface on both adjacent sides of silicon nitride according to the present invention.

### DESCRIPTION OF SYMBOLS

| | |
|---|---|
| 100: First structure | 110: First surface |
| 120: First oligonucleotide | |
| 131a, 131b: Positions spaced apart from each other | |
| 200: Second structure | 210: Second surface |
| 220: Second oligonucleotide | |
| 121, 122, 123, 124, 125, 221, 222, 223, 224, 225: | |
| Oligonucleotide | |

### DETAILED DESCRIPTION OF THE PREFERRED EMBODIMENT

While example embodiments are capable of various modifications and alternative forms, embodiments thereof are shown by way of example in the drawings and will herein be described in detail. It should be understood, however, that there is no intent to limit example embodiments to the particular fauns disclosed, but to the contrary, example embodiments are to cover all modifications, equivalents, and alternatives falling within the scope of example embodiments.

The terminology used herein is for the purpose of describing particular embodiments only and is not intended to be limiting of example embodiments. As used herein, the singular forms “a”, “an” and “the” are intended to include the plural for ms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another.

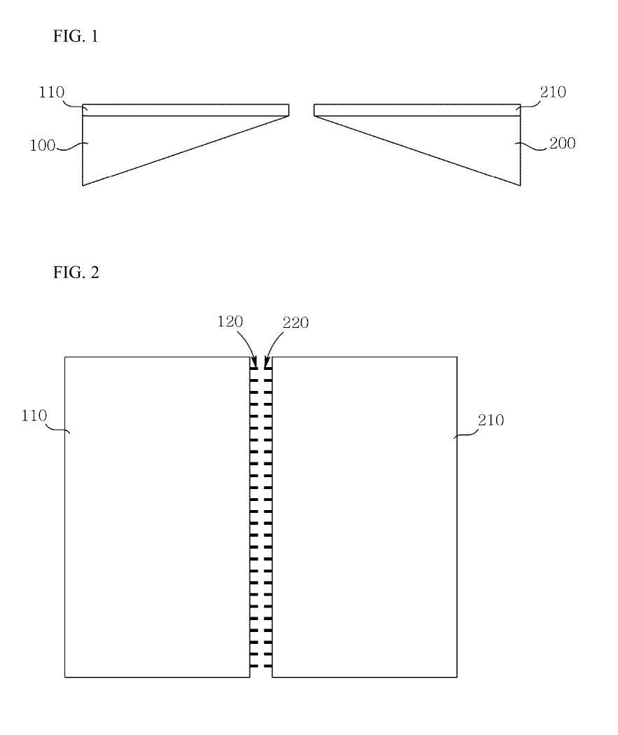

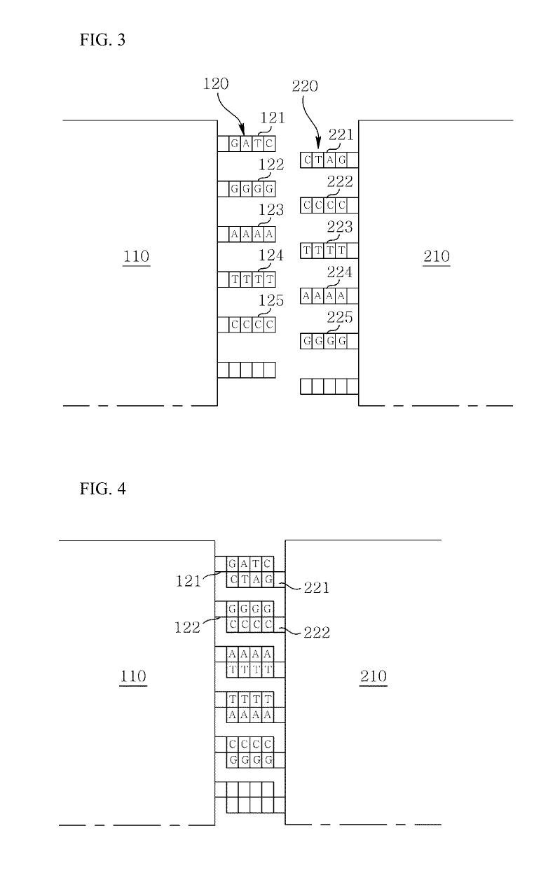

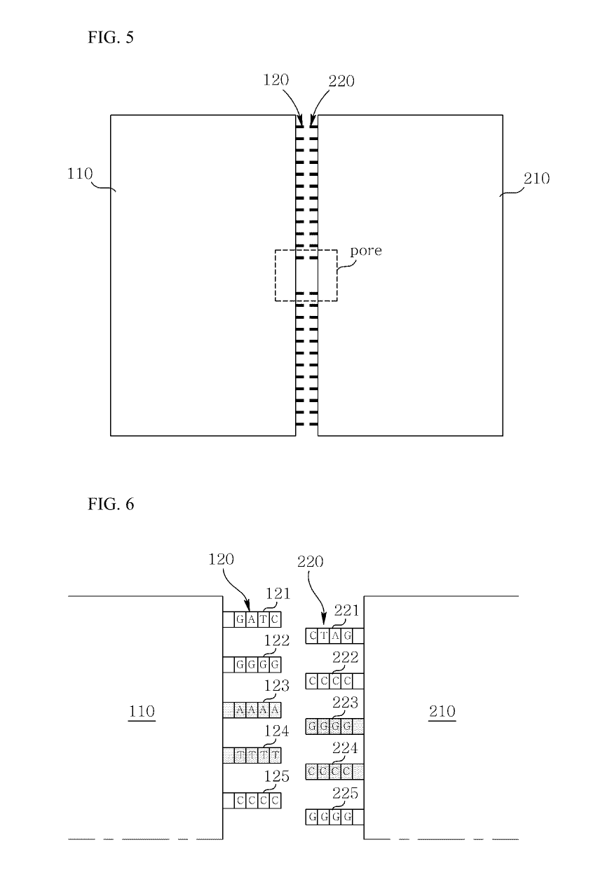

FIG. 1 is a cross-sectional view showing a structure having a surface on which nucleotides can be attached according to the present invention, FIG. 2 is a plan view showing a plurality of oligonucleotides attached on the surface of a structure according to the present invention, FIG. 3 shows an enlarged view of the plurality of oligonucleotides in FIG. 2, FIG. 4 is a mimetic view showing a plurality of oligonucleotides attached on the surface of a structure in a state of being bound to each other according to the present invention, and FIG. 5 is a mimetic view showing a process of forming a pore by removing some of a plurality of oligonucleotides bound to each other according to the present invention.

The method of forming a nanopore according to the present invention shown in the figures prepares a first structure 100 having a first surface 110 on which nucleotides can be attached and a second structure 200 having a second surface 210 on which nucleotides can be attached S110.

The first structure 100 and the second structure 200 may be an interface contained in a chamber or separating two distinguished chambers, or may be a structure containing the interface. That is, in a vessel or a well capable of containing a sample and a reaction solution, the first structure 100 and the second structure 200 may be a membrane or a wall that can separate or divide the vessel or the well, and a pore formed between the first structure 100 and the second structure 200 in a method described below may be a channel that can connect the divided or separated spaces.

The shape and material of the first structure 100 and the second structure 200 are not specially limited, and various forms known in this technical field are all included. Preferably, the first structure 100 and the second structure 200 are formed in a shape corresponding to each other as shown in FIG. 1 and the portions facing each other have a shape of a vertex rather than a flat surface so as to be suitable for making a pore of a cone shape. Therefore, further higher ion conductivity is obtained, and thus signal sensitivity can be improved.

The first structure 100 and the second structure 200 may be a substrate formed of Si, Ge, GaAs, AlAs, AlSb, GaN, GaP, GaSb, InP, Al203, SiC, InSb, CdSe, CdS, CdTe, InAs, ZnTe, ZnO, or ZnS. Alternatively, the first structure 100 and the second structure 200 may be an organic material which is a substrate formed of PVK (poly(N-vinylcabazole)), MEH-PPV (poly(2-methoxy-5-(2′-ethylhexyloxy)-p-phenylene vinylene), n-type fullerene, Polyacetylene, Polythiophene, Phthalocyanine, Poly(3-hexylthiophene), Poly(3-alkylthiophene), α-ω-hexathiophene, α-ω-di-hexyl-hexathiophene, Polythienylenevinylene, or Bis(dithienothiophene).

The first surface 110 and the second surface 210 are formed on one side of the first structure 100 and the second structure 200, respectively. Particularly, if a surface has all or only some portions on which nucleotides can be attached, it will be sufficient. The portions on which nucleotides can be attached are preferably formed on the side surfaces of the first structure 100 and the second structure 200 facing each other.

The first surface 110 and the second surface 210 may be formed of, for example, gold, or they may be a gold layer formed on a silicon nitride. In addition, the first surface 110 and the second surface 210 may be surface-modified to have a carboxyl group (—COOH), a thiol group (—SH), a hydroxyl group (—OH), a silane group, an amine group or an epoxy group using a conventional method known to a DNA chip or a protein chip so that preferably the nucleotide may be bound on a membrane of SiO₂, Al₂O₃, TiO₂, BaTiO₃, PbTiO₃, or Si₃N₄. Thickness of the first surface 110 and the second surface 210 may be 100 to 500 nm. The first surface 110 and the second surface 210 may be formed on the first structure 100 and the second structure 200 in a method of pulsed laser deposition, sputtering, chemical vapor deposition, e-beam evaporation, thermal evaporation or the like.

Next, as shown in FIG. 2, the method of forming a nanopore according to the present invention attaches a plurality of oligonucleotides on the side surface of the first surface 110 of the first structure 100 and attaches a plurality of oligonucleotides complementary to these on the side surface of the second surface 210 of the second structure 200 S120.

That is, a plurality of oligonucleotides complementary to each other is attached on the side surfaces of the first structure 100 and the second structure 200 facing each other. This is to bind the first structure 100 and the second structure 200 by binding the plurality of oligonucleotides. To this end, in the present invention, one ends of a plurality of oligonucleotides 120 may be attached on the side surface of the first surface 110 of the prepared first structure 100, and a plurality of oligonucleotides 220 having some areas complementary to each of the plurality of oligonucleotides 120 may be attached on the side surface of the second surface 210 of the prepared second structure 200.

Specifically, as shown in FIG. 3, the plurality of oligonucleotides 120 and 220 may be formed of different bases or formed of the same base. That is, all the bases in each of the oligonucleotides 121, 122, 123, 124 and 125 attached to the first surface 110 may be complementary to those in each of the oligonucleotides 221, 222, 223, 224 and 225 attached to the second surface 210, or some of the bases may be complementary to each other. In addition, all the bases in each of the oligonucleotides 121, 122, 123, 124, 125, 221, 222, 223, 224 and 225 may be the same, or the oligonucleotides may have a combination of different bases. In the present invention, lengths of the oligonucleotides 120 and 220 are preferably the same, and a pore of a desired size can be accurately formed by adjusting the length.

Preferably, it is appropriate to attach the plurality of oligonucleotides 120 attached to the first structure 100 and the plurality of oligonucleotides 220 attached to the second structure 200 at positions corresponding to each other in order to bind them. That is, it is appropriate to attach the oligonucleotides 221, 222, 223, 224 and 225 on the second surface 210 in order so as to be complementary to the oligonucleotides 121, 122, 123, 124 and 125 attached on the first surface 110. Although it is further preferable to sequentially arrange the plurality of oligonucleotides 120 and 220 in a row, the oligonucleotides 120 and 220 may be arranged to be attached in a variety of forms such as an S-shape, a zigzag shape or the like. Since it is enough for the plurality of oligonucleotides 120 and 220 to have bases complementary to each other only to be bound to each other, it is preferable to arrange the oligonucleotides 120 and 220 to form two or more layers on the first and second surfaces 110 and 210 since it may increase binding force between the first structure 100 and the second structure 200.

The plurality of oligonucleotides 120 and 220 may be attached to be spaced apart from each other by a predetermined distance or may be attached to have a densely populated side surface (in this case, the plurality of oligonucleotides 120 and 220 may be bound to each other on another side surface that is not densely populated, i.e., a side surface in the direction of the top or bottom surface in FIG. 3). Alternatively, the oligonucleotides 121, 122, 123, 124 and 125 may be formed as a set having side surfaces bound to each other and attached to the first surface 110.

The method of attaching one ends of the plurality of oligonucleotides 120 and 220 to the first and second surfaces 110 and 210 is not specially limited, and they can be attached using a method conventionally known to a DNA chip or a protein chip. To this end, it is preferable to previously embed specific functional groups at specific positions on the first and second surfaces 110 and 210 so that the oligonucleotides can be bound to.

Next, as shown in FIG. 4, the method of forming a nanopore according to the present invention binds the plurality of oligonucleotides 120 and 220 attached to the first structure 100 and the second structure 200 S130.

The plurality of oligonucleotides 120 and 220 has areas complementary to each other, and if the distance between the first structure 100 and the second structure 200 is shortened, the oligonucleotides are naturally hybridized. Accordingly, the first structure 100 and the second structure 200 may accomplish a physical structural binding. Such a binding completes a barrier or an interface, through which target nucleic acid molecules cannot pass, between the first structure 100 and the second structure 200.

Next, as shown in FIG. 5, the method of forming a nanopore according to the present invention completes forming the pore by removing some oligonucleotides among the plurality of oligonucleotides 120 and 220 bound to each other S140.

The method of removing some oligonucleotides among the plurality of the oligonucleotides 120 and 220 is not specially limited, and a variety of methods known in this technical field can be used. For example, a Transmission Electron Microscope (TEM), a Scanning Electron Microscope (SEM), a Focused Ion Beam (FIB) or an Electron beam (E-beam) can be used. A high-energy electron beam and a low-energy electron beam can be sequentially used in the same TEM.

In addition, it is possible to design a mismatch base sequence having one to three bases of artificial sequence that can be cut by a specific enzyme in some oligonucleotides among the plurality of oligonucleotides 120 and 220 and remove some of the oligonucleotides using a mismatch cleavage enzyme as the specific enzyme.

The size of removing some of the oligonucleotides can be adjusted as a user desires, and thus a pore of a desired size can be fabricated.

If the method described above is used, a structure formed with a nanopore can be fabricated. Accordingly, in another embodiment of the present invention, a structure formed with a nanopore includes a first structure 100, a second structure and a pore.

That is, the structure formed with a nanopore includes a first structure 100 having a first surface 110 attached with one ends of a plurality of oligonucleotides 120, a second structure 200 having a second surface 210 attached with a plurality of oligonucleotides 220 having all or some areas complementary to each of the plurality of oligonucleotides 120, and a pore formed by binding the plurality of oligonucleotides 120 and 220 attached to the first structure 100 and the second structure 200 and removing some of the bound oligonucleotides.

Here, the plurality of oligonucleotides 120 attached to the first structure 100 and the plurality of oligonucleotides 220 attached to the second structure 200 are preferably attached at positions corresponding to each other as described above.

In the present invention, as shown in FIG. 5, the pore is preferably formed by binding some oligonucleotides among the plurality of oligonucleotides 110 and 220 attached to the first structure 100 and the second structure 200, and some unbound oligonucleotides can be the ones that are removed.

Therefore, a pore size is determined depending on the number or size of the some bound oligonucleotides or the removed and unbound oligonucleotides, and accordingly, a user may fabricate a pore of a desired size in a simple and easy way.

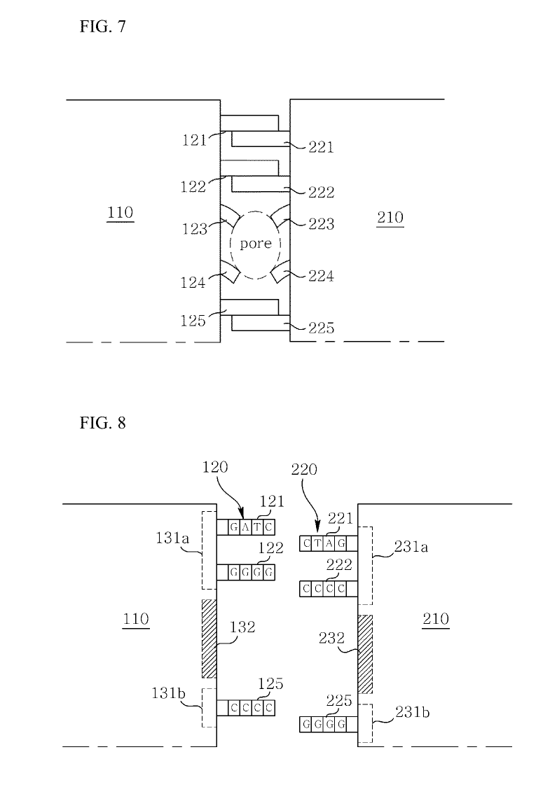

FIG. 6 is a mimetic view showing some oligonucleotides uncomplementary to each other in a state of being attached on the surface of a structure according to the present invention, and FIG. 7 is a mimetic view showing a process of forming a pore by the oligonucleotides of FIG. 6.

The method of forming a nanopore according to the present invention shown in the figures does not remove some of oligonucleotides after binding a plurality of oligonucleotides 120 and 220 complementary to each other, but some oligonucleotides 121, 122, 125 and 221, 222, 225 are arranged to have a sequence to be complementary to each other, and some other oligonucleotides 123, 124 and 223, 224 positioned between the some oligonucleotides are arranged to have a sequence not to be complementary to each other, and thus a pore can be naturally formed by binding the oligonucleotides without the process of removing some of the oligonucleotides.

To this end, the present invention performs a step of preparing a first structure and a second structure having a surface on which nucleotides can be attached S210, as described above.

Then, in the present invention, one ends of a plurality of oligonucleotides 120 are attached on the first surface 110 of the first structure 100. In addition, a plurality of oligonucleotides 221, 222 and 225 having all or some areas complementary to each of some oligonucleotides 121, 122 and 125 among the plurality of oligonucleotides 120 is attached to the second surface 210 of the prepared second structure 200. At the same time, one or more oligonucleotides 223 and 224 uncomplementary to one or more oligonucleotides 123 and 124 positioned between the some oligonucleotides 121, 122 and 125 are attached to the second surface 210 of the prepared second structure 200 S220.

Subsequently, as shown in FIG. 7 of the present invention, if the first structure 100 and the second structure 200 are positioned to be close, only the oligonucleotides 121, 122, 125 and 221, 222, 225 complementary to each other among the plurality of oligonucleotides attached to the first surface 110 and the second surface 210 are bound to each other, and some of the oligonucleotides 123, 124 and 223, 224 uncomplementary to each other are not bound to each other, and thus a pore is formed.

According to the method described above, it is possible to fabricate a structure formed with a nanopore, including a pore formed by one or more oligonucleotides 121, 122, 125 and 221, 222, 225 uncomplementary to each other.

The structure formed with a nanopore of the present invention described above may include a first structure 100 having a first surface 110 attached with one ends of a plurality of oligonucleotides 120, a second structure 200 having a second surface 210 attached with a plurality of oligonucleotides 221, 222 and 225 having all or some areas complementary to each of some oligonucleotides 121, 122 and 125 among the plurality of oligonucleotides 120 and one or more oligonucleotides 223 and 224 uncomplementary to one or more oligonucleotides 123 and 124 positioned between the some oligonucleotides 121, 122 and 125, and a pore formed by one or more oligonucleotides 123, 124 and 223, 224 uncomplementary to each other, in which complementary oligonucleotides 121, 122, 125 and 221, 222, 225 among the plurality of oligonucleotides attached to the first structure 100 and the second structure 200 are bound to each other.

Here, it is preferable to position the one or more uncomplementary oligonucleotides 223 and 224 between the complementary oligonucleotides 221, 222 and 225 attached to the second surface 210

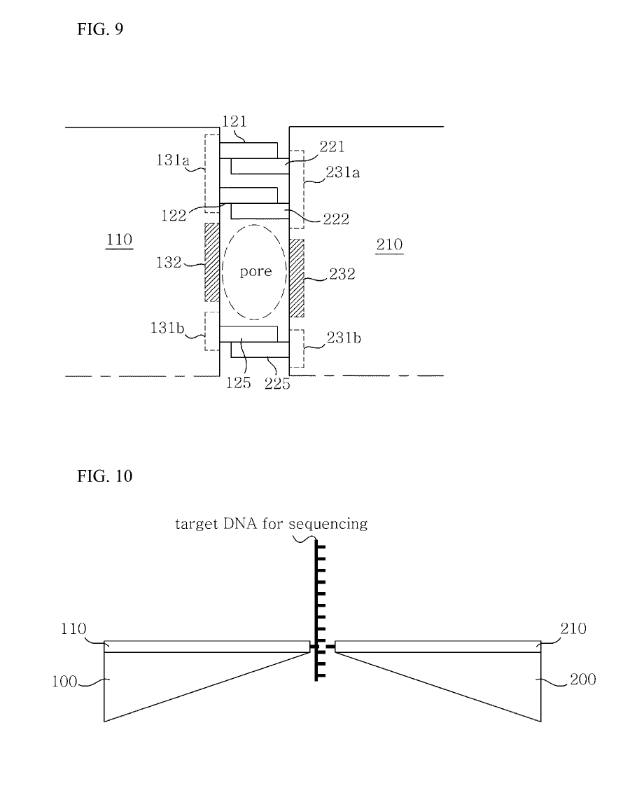

FIG. 8 is a mimetic view showing oligonucleotides complementary to each other in a state of being attached at positions apart from each other on the surface of a structure according to the present invention, and FIG. 9 is a mimetic view showing a process of forming a pore by the oligonucleotides of FIG. 8.

The method of forming a nanopore according to the present invention shown in the figures does not use oligonucleotides uncomplementary to each other from the beginning and forms a pore using only oligonucleotides 121, 122, 125 and 221, 222, 225 complementary to each other.

To this end, the present invention performs a step of preparing a first structure and a second structure having a surface on which nucleotides can be attached S310, as described above.

Then, the present invention performs a step of attaching one ends of a plurality of oligonucleotides 121, 122 and 125 at positions 131a and 131b spaced apart from each other on one side surface, i.e., the first surface 110 of the prepared first structure 100, and attaching a plurality of oligonucleotides 221, 222 and 225 having all or some areas complementary to each of the plurality of oligonucleotides 221, 222 and 225 at positions 231a and 21b on one side surface, i.e., the second surface 210 of the prepared second structure 200 corresponding to positions 131a and 131b of the first structure 100 spaced apart from each other S320.

Subsequently, as shown in FIG. 9 of the present invention, if the first structure 100 and the second structure 200 are moved to be close, a plurality of oligonucleotides 121, 122, 125 and 221, 222, 225 attached at positions 131a, 131b and 231a, 231b spaced apart from each other on the first surface 110 and the second surface 210 are bound to each other, and a pore is naturally formed in the areas 132 and 232 between the positions 131a, 131b and 231a, 231b spaced apart from each other.

If the method described above is used, it is possible to fabricate a structure formed with a nanopore including a pore formed between a plurality of complementary oligonucleotides 121, 122, 125 and 221, 222, 225.

The structure formed with a nanopore of the present invention described above may include a first structure 100 having a surface attached with one ends of a plurality of oligonucleotides 121, 122 and 125 at positions 131a and 131b spaced apart from each other, a second structure 200 having a surface attached with a plurality of oligonucleotides 221, 222 and 225 having all or some areas complementary to each of the plurality of oligonucleotides 121, 122 and 125 at positions 231a and 231b corresponding to the positions 131a and 131b spaced apart from each other on the first structure 100, and a pore formed between the positions 131a, 131b and 231a, 231b spaced apart from each other, by attaching the plurality of oligonucleotides 121, 122, 125 and 221, 222, 225 on the first structure 100 and the second structure 200 bound to each other.

Although the oligonucleotides are described as an example in this specification, the present invention is characterized by using the binding between the oligonucleotides complementary to each other, and thus it is apparent to those skilled in the art that using the oligonucleotides is also included in the claims of the present invention.

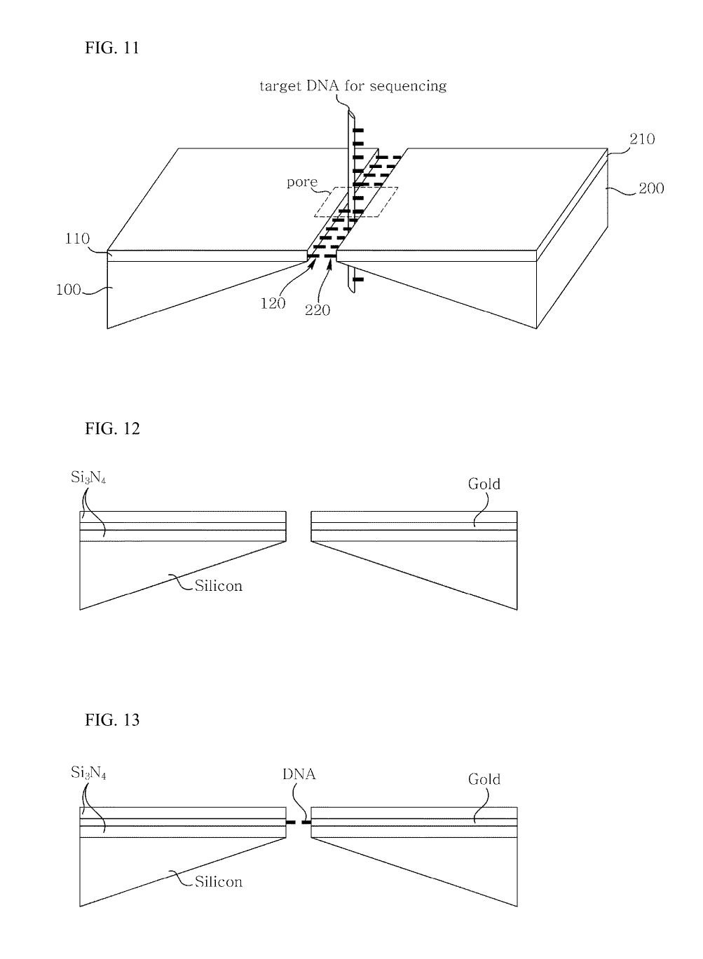

FIG. 10 is a cross-sectional view showing a process of sequencing target DNA using a structure formed with a pore according to an embodiment of the present invention, and FIG. 11 is a perspective view of FIG. 10.

As shown in the figures, if target nucleic acid molecules pass through the nanopore using the structure formed with the nanopore fabricated by the fabrication method described above, the target nucleic acid molecules can be detected and analyzed.

To this end, a sample containing DNA passing through the nanopore can be dissolved in an electrically conductive solvent and prepared in a fluid state, and at this point, a certain convenient and electrically conductive solvent can be used. The solvent is a water-based solvent, and it can be pure water or water containing one or more additive materials, such as water containing a buffer agent or salt (e.g., potassium chloride). Preferably, the solvent is an ionized buffer solution such as 1 M KCl, 10 Mm Tris-HCl or the like. In addition, pH of the fluid sample is typically about 6.0 to 9.0.

In addition, the present invention also provides a nucleic acid molecule detection apparatus using a nanopore, including a structure formed with the nanopore, an electrode for applying voltage to the nanopore of the structure, and a measurement unit for measuring an electrical signal generated when a sample containing DNA passes through the nanopore.

The nanopore used in the apparatus of the present invention means a structure having a channel or a pore (hole) with a diameter of a nanometer scale, and the nanopore is a part of a nanopore detection apparatus of a publicized technical configuration including a nanopore sensor. The configuration and method of the nucleic acid molecule detection apparatus using a nanopore are different from the method of forming a nanopore described above and the nanopore formed according to the method only in the structure and material, and publicized techniques can be applied to the other parts thereof.

That is, generally, the nanopore detection apparatus detects a target material in a fluid flowing through the nanopore by applying electric fields passing through the nanopore and monitoring changes in the current passing through the nanopore. Amplitude of the current passing through the nanopore is monitored while the fluid flows, and changes in the amplitude relate to passage of the target material through the nanopore, and thus the target material can be efficiently detected from the measured amplitude value of current.

Further specifically, in the present invention, a pore of a desired size can be accurately formed by adjusting the length of a plurality of oligonucleotides attached on the surface of a structure. According to a conventional technique existing prior to the application of the present invention, it is unable to fabricate a nanopore having a diameter less than 10 nm. However, according to the present invention, a nanopore can be fabricated to have a diameter less than 10 nm, preferably less than 5 nm, and further preferably even less than 1 nm, and thus a detection apparatus having a superior resolution can be provided.

In some cases, the apparatus of the present invention may further include a sample storage chamber connected to the structure and storing a sample to be put into the nanopore. The sample is a fluid material containing a PCR product, i.e., DNA amplified through a PCR method, and particularly, the DNA may be double or single stranded DNA having a size less than 1 kbp. In addition, although the sample storage chamber may be implemented in a configuration for storing a sample injected from outside, it may be implemented in a configuration for creating and storing a desired sample using a publicized DNA amplifier, e.g., a PCR chip. At this point, in some cases, the sample storage chamber may be implemented to be connected to the DNA amplifier connected through a micro channel having a diameter of a nanopore size and supplied with a sample containing DNA.

Meanwhile, although it is not specifically described, functional components of the present invention described above can be implemented in process-on-a-chip or lab-on-a-chip using publicized Microfluidic units and MEMS devices.

The present invention will be further clearly understood from the embodiments described below, and the embodiments described below are for illustrative purpose only and are not construed to limit the scope of the present invention by the accompanying claims.

### Embodiment 1

### A Structure Formed with a Nanopore Having a Surface of a Gold Layer at One End

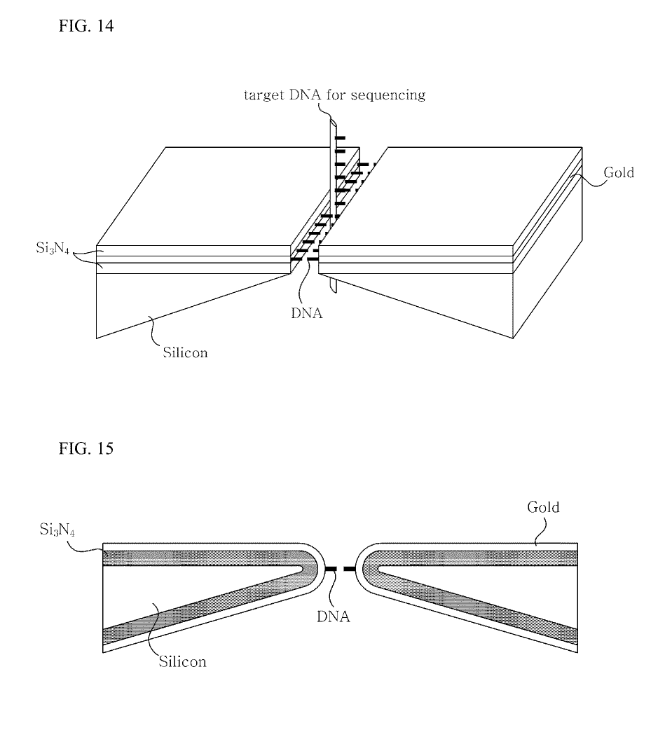

FIG. 12 is a cross-sectional view showing a silicon structure having a gold surface on which nucleotides can be attached according to the present invention, FIG. 13 is a cross-sectional view showing DNA oligonucleotides attached to the structure of FIG. 12, and FIG. 14 is a perspective view showing a process of sequencing target DNA using the structure of FIG. 12.

That is, first and second structure blocks are fabricated as shown in FIG. 12 by sharply cutting one side of a silicon material and evaporating silicon nitride, gold and silicon nitride in order on the other side adjacent to the sharpened vertex.

A single stranded DNA is attached on a side surface of the gold layers of the first and second structure blocks facing each other, and the first and second structure blocks are connected through DNA bindings (FIG. 13).

Next, a nanopore having a certain size is formed by removing some of the bindings of the connected DNS binding unit.

A sequence is determined from the signal generated by passing a target DNA through the nanopore using the structure formed with the nanopore as described above (FIG. 14).

### Embodiment 2

### A Structure Formed with a Nanopore Having a Surface of a Gold Layer at Both Ends

FIG. 15 is a cross-sectional view showing a structure formed with a gold surface on both adjacent sides of silicon nitride according to the present invention.

In this embodiment, first and second structure blocks are fabricated as shown in FIG. 15 by sharply cutting one side of a silicon material and evaporating silicon nitride and gold and in order on two sides adjacent to the sharpened vertex.

A pore is formed by attaching DNA to each of the blocks, binding the first and second structure blocks together, and removing some of the bindings in the same manner as described in embodiment 1.

The present invention is effective in that a pore of a desired size can be accurately formed by adjusting the length of a plurality of oligonucleotides attached on the surface of a structure. According to a conventional technique existing prior to the application of the present invention, it is unable to fabricate a nanopore having a diameter less than 10 nm. However, according to the present invention, a nanopore can be fabricated to have a diameter less than 10 nm, preferably less than 5 nm, and farther preferably even less than 1 nm.

Furthermore, the present invention may form a pore in a simple and easy way by attaching a plurality of oligonucleotides on the surfaces of the first and second structures and then binding the first and second structures.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A method of forming a nanopore, the method comprising the steps of:
preparing a first structure and a second structure having a surface on which nucleotides can be attached;
attaching one ends of a plurality of oligonucleotides on the surface of the prepared first structure, and attaching one ends of a plurality of oligonucleotides having all or some areas complementary to each of the plurality of oligonucleotides on the surface of the prepared second structure; and
binding the plurality of oligonucleotides attached to the first structure and the second structure to each other.

2. The method according to claim 1, wherein the step of attaching includes the steps of attaching one ends of the plurality of oligonucleotides on the surface of the prepared first structure, and attaching one ends of the plurality of oligonucleotides having some areas complementary to each of the plurality of oligonucleotides on the surface of the prepared second structure, and after the step of binding each other, the step of removing some oligonucleotides among the plurality of oligonucleotides bound to each other is further provided.

3. The method according to claim 2, wherein some oligonucleotides among the plurality of oligonucleotides include a base sequence that can be cut by a specific enzyme in the oligonucleotides.

4. The method according to claim 1, wherein the step of attaching includes the steps of attaching one ends of the plurality of oligonucleotides on the surface of the prepared first structure, and attaching a plurality of oligonucleotides having all or some areas complementary to each of some oligonucleotides among the plurality of oligonucleotides and one or more oligonucleotides uncomplementary to oligonucleotides positioned between the some oligonucleotides on the surface of the prepared second structure, and
the step of binding to each other is the step of binding the complementary oligonucleotides among the plurality of oligonucleotides attached to the first structure and the second structure to each other.

5. The method according to claim 4, wherein the step of binding oligonucleotides to each other is binding oligonucleotides complementary to each other among the plurality of oligonucleotides attached to the first structure and the second structure and forming the pore by the one or more uncomplementary oligonucleotides.

6. The method according to claim 1, wherein the step of attaching includes the steps of attaching one ends of the plurality of oligonucleotides at positions spaced apart from each other on one surface of the prepared structure, and
attaching a plurality of oligonucleotides having all or some areas complementary to each of the plurality of oligonucleotides on one surface of the prepared second structure corresponding to the positions spaced apart from each other on the first structure.

7. The method according to claim 6, wherein the step of binding the oligonucleotides to each other is binding oligonucleotides complementary to each other among the plurality of oligonucleotides attached to the first structure and the second structure and forming the pore at the spaced position.

8. A structure formed with a nanopore, the structure comprising:
a first structure having a surface attached with one ends of a plurality of oligonucleotides;
a second structure having a surface attached with a plurality of oligonucleotides having all or some areas complementary to each of the plurality of oligonucleotides; and
a pore formed by binding the plurality of oligonucleotides attached to the first structure and the second structure.

9. The structure according to claim 8, wherein the plurality of oligonucleotides attached to the first structure and the second structure is bound to each other, and the pore is formed by removing some of the bindings.

10. The structure according to claim 9, wherein the plurality of oligonucleotides attached to the first structure and the plurality of oligonucleotides attached to the second structure are attached at positions corresponding to each other.

11. The structure according to claim 8, wherein the second structure has a surface attached with a plurality of oligonucleotides having all or some areas complementary to each of some oligonucleotides among the plurality of oligonucleotides and one or more oligonucleotides uncomplementary to oligonucleotides positioned between the some oligonucleotides, and
the pore is formed by the one or more uncomplementary oligonucleotides, while the complementary oligonucleotides among the plurality of oligonucleotides attached to the first structure and the second structure are bound to each other.

12. The structure according to claim 11, wherein the one or more uncomplementary oligonucleotides are positioned between the complementary oligonucleotides attached to the second structure.

13. The structure according to claim 8, wherein the first structure has one surface attached with one ends of the plurality of oligonucleotides at positions spaced apart from each other, and
the second structure has a surface attached with a plurality of oligonucleotides having all or some areas complementary to each of the plurality of oligonucleotides at positions corresponding to the positions spaced apart from each other on the first structure.

14. The structure according to claim 13, wherein the plurality of oligonucleotides attached to the first structure and the second structure is bound to each other, and the pore is formed between the positions spaced apart from each other.

15. A nucleic acid molecule detection apparatus using a nanopore, the apparatus comprising:
a structure formed with the nanopore of claim 8;
an electrode for applying voltage to the nanopore of the structure; and
a measurement unit for measuring an electrical signal generated when a sample containing DNA passes through the nanopore.
